(19) United States
(12) Patent Application Publication
(10) Pub. No.: US 2021/0387146 A1
(43) Pub. Date: Dec. 16, 2021

(54) COLLECTION OF CELLS FROM BIOLOGICAL FLUID

(71) Applicant: DUKE UNIVERSITY, Durham, NC (US)

(72) Inventors: Michael Wong, Durham, NC (US); Anna Matthews, Durham, NC (US); Brandon Tramontana, Durham, NC (US); Nicolas Swischuk, Durham, NC (US); Alejandro Pino, Durham, NC (US); Scott Shofer, Durham, NC (US)

(21) Appl. No.: 17/345,500

(22) Filed: Jun. 11, 2021

Related U.S. Application Data
(60) Provisional application No. 63/037,705, filed on Jun. 11, 2020.

(51) Int. Cl. B01D 69/02; A61B 10/00; B01D 65/00

(52) U.S. Cl. CPC B01D 69/02; A61B 10/0045; B01D 2325/48; B01D 2325/36; B01D 2325/02; B01D 65/003

(57) ABSTRACT

A biological filtering device includes a fluid inlet for receiving a biological fluid, a human cell filter coupled to the fluid inlet and having a pore size to capture human cells, a bacteria cell filter having a pore size to capture bacteria cells, a fluid connector fluidically connecting the human cell filter to the bacteria cell filter, and a fluid outlet coupled to the bacteria cell filter for discharging the biological fluid. In some cases, the biological filtering device may include an additional human cell filter and/or an additional bacteria cell filter. A method of using a biological filtering device includes fluidically coupling the biological filtering device between a syringe and a biological fluid storage unit, inserting a catheter into a patient, and pulling biological fluid, via the syringe, from the patient, through the human cell filter and the bacteria cell filter, and into the biological fluid storage unit.

### BACKGROUND

Pleural effusion is the build-up of excess fluid between layers of the pleura outside the lungs (e.g., between the lung and chest). Causes of pleural effusion include, but are not limited to, heart failure, pulmonary embolism, cirrhosis, pneumonia, cancer, kidney disease, tuberculosis, and inflammatory disease. Build-up of fluid can also occur in other areas of a patient's body. Treatment for build-up of excess fluid in a patient's body includes chemotherapy, in-dwelling catheters, pleural apposition, or repeated thoracentesis. For most of the population, a diagnosis of malignant pleural effusion is the first sign of the disease, and the prognosis is usually between three to twelve months. Rapid identification and treatment of the cause of the pleural effusion (or cause of a build-up of excess fluid in other areas of a patient's body) greatly improves the prognosis, however, accurate diagnosis of the underlying cause of the pleural effusion remains problematic.

One current solution is a thoracentesis procedure, in which the pleural space of a patient is catheterized and manually removed (e.g., via a syringe) by a physician. Fluid samples of the biological fluid are collected for further analysis during the thoracentesis procedure, however, due to the relatively small sample size (e.g., 100 mL of the total 1 L to 1.5 L amount of biological fluid that is removed during the thoracentesis procedure), an accurate diagnosis of the underlying cause of the pleural effusion is not achieved, ultimately resulting in a lower prognosis for the patient.

### BRIEF SUMMARY

Biological filtering devices and methods of using those devices are provided. A biological filtering device as described herein makes it possible to screen a larger percentage or even all of the biological fluid that is removed from a patient during certain procedures, such as a thoracentesis procedure. By measuring a larger percentage of the biological fluid withdrawn from a patient, physicians are able to offer more accurate diagnosis and treatment for the underlying cause of the excess biological fluid; diagnosis and treatment that may otherwise not be possible using the relatively small percentage of biological fluid that is examined using current devices and methods. Furthermore, by filtering out the human cells and bacteria cells from the biological fluid, the volume of material needed for testing, as well as the costs associated with that testing (e.g., shipping and handling costs of biohazardous materials) is greatly reduced.

A biological filtering device includes a fluid inlet for receiving a biological fluid, a human cell filter coupled to the fluid inlet and having a pore size to capture human cells, a bacteria cell filter having a pore size to capture bacteria cells, a fluid connector fluidically connecting the human cell filter to the bacteria cell filter, and a fluid outlet coupled to the bacteria cell filter for discharging the biological fluid.

In some cases, the pore size of the bacteria cell filter is equal to or greater than 0.1 microns and less than 0.51 microns. In some cases, the pore size of the human cell filter is 10 equal to or greater than 2 microns and less than 15.1 microns. In some cases, the human cell filter and the bacteria cell filter are hydrophilic. In some cases, the fluid inlet and the fluid outlet include Luer-Lock connections. In some cases, the fluid connector is coupled to the human cell filter and the bacteria cell filter. In some cases, the biological filtering device further includes a second human cell filter coupled to the fluid connector between the human cell filter and the bacteria cell filter and having a pore size to capture human cells, and the pore size of the second human cell filter is smaller than the pore size of the human cell filter. In some cases, the biological filtering device further includes a second bacteria cell filter coupled to the fluid connector between the human cell filter and the bacteria cell filter and having a pore size to capture bacteria cells, and the pore size of the second bacteria cell filter is larger than the pore size of the bacteria cell filter. In some cases, the human cell filter and the bacteria cell filter each include a filter membrane, a casing formed around the filter membrane, and a gasket fluidically sealing the casing around the filter membrane. In some cases, the biological filtering device further includes a pressure release valve that is configured to reverse flow of biological fluid during a biological fluid removal procedure.

A biological fluid removal and filtering device includes a syringe for withdrawing biological fluid from a patient, a biological filtering device fluidically coupled to the syringe, and a biological fluid storage unit fluidically coupled to the biological filtering device. The biological filtering device includes a fluid inlet for receiving a biological fluid, a human cell filter coupled to the fluid inlet and having a pore size to capture human cells, a bacteria cell filter having a pore size to capture bacteria cells, a fluid connector connecting the human cell filter to the bacteria cell filter, and a fluid outlet coupled to the bacteria cell filter for discharging the biological fluid. In some cases, during use, the syringe is used to pull biological fluid from a patient, through human cell filter and the bacteria cell filter, and into the biological fluid storage unit. In some cases, the biological fluid removal and filtering device further includes a catheter attached to the syringe, and the catheter is used to access biological fluid within a patient. In some cases, the biological fluid removal and filtering device includes a one-way valve preventing fluid from re-entering the patient.

A method of using a biological filtering device includes fluidically coupling the biological filtering device between a syringe and a biological fluid storage unit, inserting a catheter into an area of a patient having biological fluid, and pulling biological fluid, via the syringe, from the patient, through the human cell filter and the bacteria cell filter, and into the biological fluid storage unit.

In some cases, the biological filtering device further includes a human cell filter pressure sensor coupled to the human cell filter, and the method further includes monitoring a pressure reading of the human cell filter pressure sensor. In some cases, the method further includes, upon the human cell filter pressure sensor indicating a predetermined pressure, changing the human cell filter. In some cases, the biological filtering device further includes a pressure relief valve coupled to the human cell filter, and the method further includes, upon the human cell filter pressure sensor indicating a predetermined pressure, releasing pressure in the human cell filter via the pressure relief valve. The pressure sensor and pressure relief valve have the potential to aid in diverting flow around the filter in the event that the filter becomes clogged.

In some cases, the biological filtering device further includes a bacteria cell filter pressure sensor coupled to the bacteria cell filter, and the method further includes monitoring a pressure reading of the bacteria cell filter pressure sensor. In some cases, the method further includes, upon the bacteria cell filter pressure sensor indicating a predetermined pressure, changing the bacteria cell filter. In some cases, the biological filtering device further includes a pressure relief valve coupled to the bacteria cell filter, and the method further includes, upon the bacteria cell filter pressure sensor indicating a predetermined pressure, releasing pressure in the bacteria cell filter via the pressure relief valve.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIGS. 1A and 1B illustrate perspective views of a biological filtering device.

FIGS. 1C, 1D, and 1E illustrate exploded views of a biological filtering device.

FIG. 1F illustrates a perspective view of a casing of a biological filtering device.

FIG. 2 illustrates a biological fluid removal and filtering device to remove fluid from a patient's lung.

FIG. 3 illustrates a method of using a biological fluid removal and filtering device.

FIGS. 4A-4D illustrate an inlet side casing of a biological filtering device.

FIGS. 5A-5D illustrate a middle casing of a biological filtering device.

FIGS. 6A-6D illustrate an outlet side casing of a biological filtering device.

FIGS. 7A and 7B illustrate perspective views of a biological filtering device.

FIGS. 7C, 7D, and 7E illustrate exploded views of a biological filtering device.

FIG. 7F illustrates a perspective view of a casing of a biological filtering device.

FIGS. 8A, 8B, and 8C illustrate a middle casing of a biological filtering device.

### DETAILED DESCRIPTION

Biological filtering devices and methods of using those devices are provided. A biological filtering device as described herein makes it possible to screen a larger percentage or even all of the biological fluid that is removed from a patient during certain procedures, such as a thoracentesis procedure. By screening a larger percentage of the biological fluid withdrawn from a patient, physicians are able to offer more accurate diagnosis and treatment for the underlying cause of the excess biological fluid; diagnosis and treatment that may otherwise not be possible using the relatively small percentage of biological fluid that is examined using current devices and methods. Furthermore, by filtering out the human cells and bacteria cells from the biological fluid, the volume of material needed for testing, as well as the costs associated with that testing (e.g., shipping and handling costs of biohazardous materials) is greatly reduced.

Biological filtering devices and methods described herein are useful in combination with procedures that remove excess bodily fluid. Standard treatment for excess bodily fluid, such as pleural effusion, requires drainage of that fluid. Fluid drainage is accomplished, for example, via thoracentesis (or similar procedure for the area of the body having the excess biological fluid) or placement of a tube in the area of the body having the excess biological fluid, and then draining the biological fluid from that area of the body. The typical volume of biological fluid that is drained during a thoracentesis procedure ranges from 1 L to 1.5 L, however, only about 100 mL of that biological fluid is collected; and only about 10 mL of that is cultured, with the remaining 60 mL to 90 mL being sent for cytological analysis. Due to the relatively small sample size, there is often a poor yield of bacteria (e.g., 20% to 30% in large cohorts), and antibiotic treatment is empirical on the basis of local knowledge and clinical judgement. This lack of a specific microbiological diagnosis leads to nonspecific and broad-spectrum antibiotic treatment, which in turn risks inaccurate management and poor outcomes, including the development of antibiotic resistant bacteria, complications of antibiotic therapies, and surgical intervention.

The biological filtering devices and methods remedy these problems by providing a multi-stage filter that can be used to filter human cells (e.g., leukocytes) and bacteria cells from the entirety of the biological fluid that is drained from a patient. This also provides additional advantages, such as a small amount of material (e.g., a few relatively small filter media) that is needed for microbiological diagnosis. Indeed, microbiological diagnosis of biological material often requires that biological material be sent away for microbiological testing, and the biological filtering devices and methods disclosed herein provide a way to sample the entirety of the biological fluid that is drained from a patient with only a small amount of material to actually be sent away for microbiological testing. It should be understood that while examples described herein describe pleural fluid that is collected via a thoracentesis procedure, other types of biological fluid, including but not limited to, peritoneal fluid from paracentesis procedures, urine samples, cerebrospinal fluid from lumbar punctures, cisternal punctures, and/or ventricular punctures, and bronchoalveolar lavage fluid, can also be used with the biological filtering devices and methods described herein.

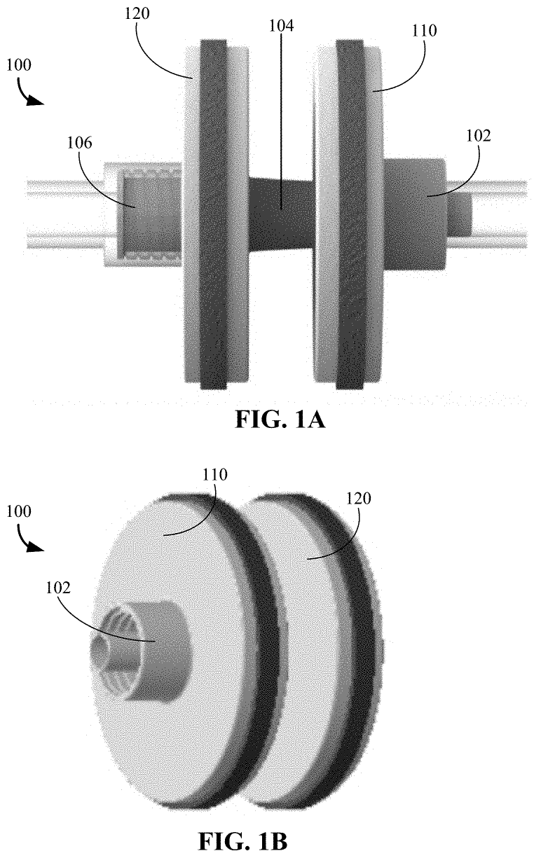

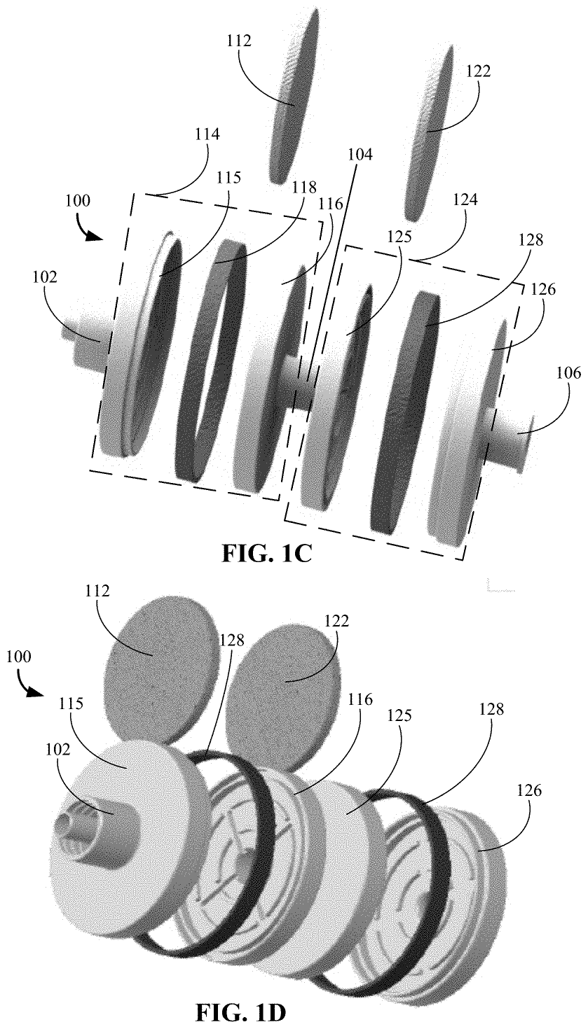

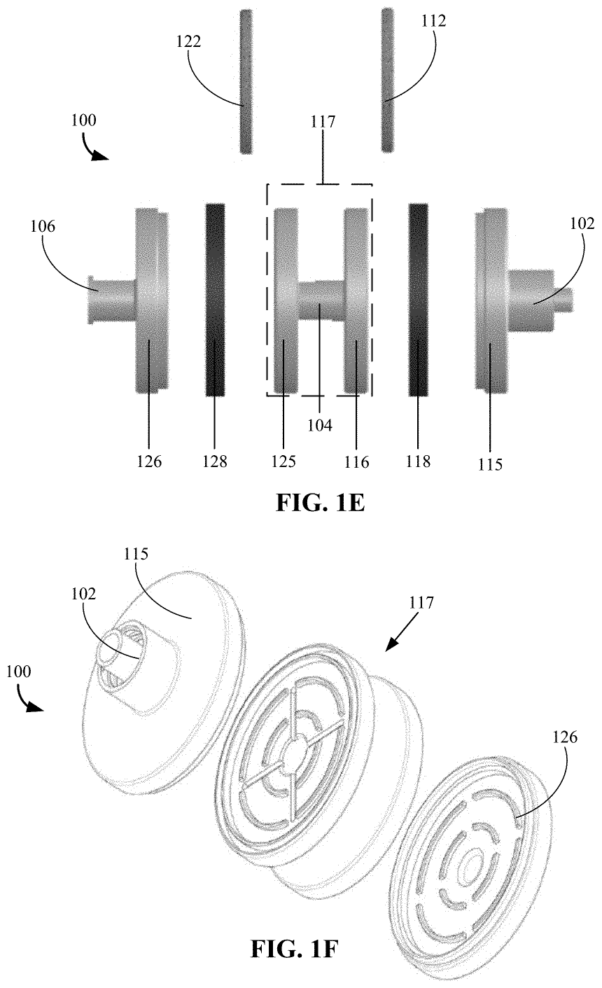

FIGS. 1A and 1B illustrate perspective views of a biological filtering device. FIGS. 1C, 1D, and 1E illustrate exploded views of a biological filtering device. FIG. 1F illustrates a perspective view of a casing of a biological filtering device. Referring to FIGS. 1A-1F, a biological filtering device 100 includes a fluid inlet 102 for receiving biological fluid, a human cell filter 110 coupled to the fluid inlet 102 and having a pore size to capture human cells, a bacteria cell filter 120 having a pore size to capture bacteria cells, a fluid connector 104 fluidically connecting the human cell filter 110 and the bacteria cell filter 120, and a fluid outlet 106 for discharging the biological fluid.

During use, the flow of biological fluid enters a biological filtering device 100 via a fluid inlet 102, flows through the biological filtering device 100, and exits the biological filtering device 100 via a fluid outlet 106. In some cases, upstream refers to a position within the flow of the biological filtering device 100 that is closer to the fluid inlet 102 relative to another position within the flow of the biological filtering device 100. In some cases, downstream refers to a position within the flow of the biological filtering device 100 that is closer to the fluid outlet 106 relative to another position within the flow of the biological filtering device 100.

In some cases, as illustrated in FIGS. 1A-1F, the fluid connector 104 is physically (e.g., attached via structure and fluidically) coupled to the human cell filter 110 and the bacteria cell filter 120. In some cases, the biological filtering device 100 further includes a second human cell filter coupled to the fluid connector 104 between the human cell filter 110 and the bacteria cell filter 120. The pore size of the second human cell filter is smaller than the pore size of the human cell filter 110 (which is why the second human cell filter is positioned downstream of the human cell filter 110). In some cases, the biological filtering device 100 further includes a second bacteria cell filter coupled to the fluid connector 104 between the human cell filter 110 and the bacteria cell filter 120. The pore size of the second bacteria cell filter is larger than the pore size of the bacteria cell filter 120 (which is why the second bacteria cell filter is positioned upstream of the bacteria cell filter 120). In some cases, the biological filtering device 100 can include two or more of human cell filters and/or bacteria cell filters. Indeed, due to the structure of the casing for the filters, as many filters with as many different pore sizes as is desired can be added to the biological filtering device 100, as is explained in further detail below.

The order in which the filters are placed depends on the pore sizes those filters; filters with the largest pore size will be added in the most upstream position (e.g., such that the biological fluid passing through the biological filtering device 100 will pass through filters with the largest pore size first) and filters with the smallest pore size will be added in the most downstream position (e.g., such that the biological fluid passing through the biological filtering device 100 will pass through filters with the smallest pore size last). By placing filters in order (e.g., upstream/downstream positioning) depending on their pore size, each filter will collect the intended cells (e.g., certain types of human and/or bacteria cells) for that filter, with filters having a larger pore size allowing the smaller cells to be collected downstream by filters having a smaller pore size.

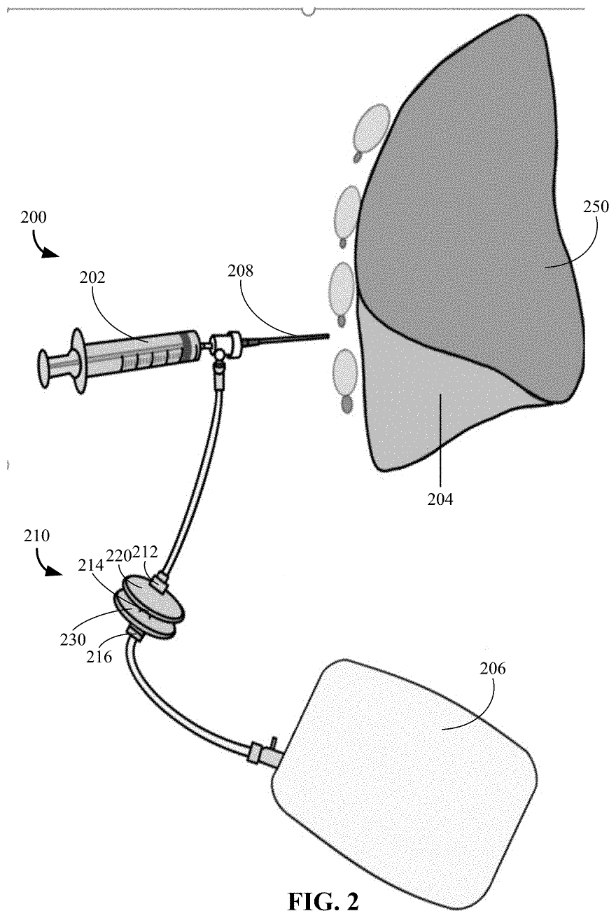

Referring specifically to FIGS. 1C-1F, in some cases, when assembled, both the human cell filter 110 and the bacteria cell filter 120 each include a filter membrane 112, 122, a casing 114, 124 formed around the filter membrane 112, 122, and a gasket 118, 128 fluidically sealing the casing 114, 124 around the filter membrane 112, 122. In other words, the gasket 118, 128 provides a fluid proof seal around its corresponding filter 110, 120. In some cases, a pressure relief valve is configured to reverse flow and/or release pressure of biological fluid during a biological fluid removal procedure, as is explained in further detail below with respect to FIG. 2.

In some cases, the casing 114 of the human cell filter 110 includes an upstream end 115 that is attached to the fluid inlet 102 and a downstream end 116 that is attached to the fluid connector 104. In some cases, the casing 124 of the bacteria cell filter 120 includes an upstream end 125 that is attached to the fluid connector 104 and a downstream end 126 that is attached to the fluid outlet 106. In some cases, the fluid connector 104 is attached to both the downstream end 116 of the casing 114 of the human cell filter 110 and the upstream end 125 of the casing 124 of the bacteria cell filter 120 (hereinafter referred collectively as a middle casing 117). In this way, in order to add an additional human cell filter, a user can simply add a second middle casing upstream of the middle casing 117 (along with the appropriate filter media and gasket); and to add an additional bacteria cell filter, a user can simply add a second middle casing downstream of the middle casing 117 (along with the appropriate filter media and gasket).

In some cases, the pore size of the bacteria cell filter membrane 122 is equal to or greater than 0.1 micron and less than 0.51 microns. In some cases, the pore size of the bacteria cell filter membrane 122 is 0.22 microns. In some cases, the pore size of the bacteria cell filter membrane 122 is 0.3 microns. In some cases, the bacteria cell filter membrane 122 is made of polytetrafluoroethylene (PTFE). In some cases, the bacteria cell filter membrane 122 is made of polyether sulfone (PES).

In some cases, the pore size of the human cell filter membrane 112 is greater than 2 microns and less than 15.1 microns. In some cases, the pore size of the human cell filter membrane 112 is 4 microns. In some cases, the pore size of the human cell filter membrane 112 is 10 microns. In some cases, the human cell filter membrane 112 has a pore size that allows passage of platelet, bacteria, and red blood cells, but retains leukocytes. In some cases, the platelet, bacteria, and red blood cells that pass through the (first) human cell filter 110 are captured by additional human cell filter(s) and/or bacteria cell filter(s) downstream. In some cases, the human cell filter membrane 112 is made of a mixed cellulose ester (MCE) membrane; in some cases, MCE is made of cellulose acetate and cellulose nitrate. In some cases, the human cell filter 110 is biologically inert. In some cases, the human cell filter membrane 112 is compatible with dilute bases, acids, and organic compounds such as formaldehyde and alcohol. In some cases, the filter membrane 112 of the human cell filter 110 and/or the filter membrane 122 of the bacteria cell filter 120 are hydrophilic.

In the case of multiple human cell filters and/or bacteria cell filters, each of the respective filter membranes may have a pore size falling within the above described ranges, with the larger pore size being upstream of the smaller pore size. Furthermore, the exact pore size of each filter membrane depends on the specific type of human and/or bacteria cells that are desired to be collected, based on the known sizes of those human and/or bacteria cells. It should be understood that the pore size of the human cell filter 110 and the bacteria cell filter 120 refers to the pore size of the filter membrane 112, 122 for that filter. In some cases, the diameter (D) of each filter membrane may be between 25 mm to 135 mm, depending upon the type of application the filter membrane is used (e.g., the amount and type of biological fluid that will pass through the filter membrane).

FIG. 2 illustrates a biological fluid removal and filtering device to remove fluid from a patient's lung 250. Referring to FIG. 2, a biological fluid removal and filtering device 200 includes a syringe 202 for withdrawing biological fluid 204 from a patient, a biological filtering device 210 fluidically coupled to the syringe 202, and a biological fluid storage unit 206 fluidically coupled to the biological filtering device 210. The syringe 202 is upstream of the biological filtering device 210 and the biological fluid storage unit 206, the biological filtering device 210 is downstream of the syringe 202 and upstream of the biological fluid storage unit 206, and the biological fluid storage unit 206 is downstream of the syringe 202 and the biological filtering device 210. In some cases, the biological fluid storage unit 206 is a medical bag; in other cases, the biological fluid storage unit 206 is a larger storage unit for biohazardous waste.

The biological filtering device 210 includes a fluid inlet 212 for receiving the biological fluid 204, a human cell filter 220 coupled to the fluid inlet 212 and having a pore size to capture human cells, a bacteria cell filter 230 having a pore size to capture bacteria cells, a fluid connector 214 fluidically connecting the human cell filter 220 and the bacteria cell filter 230, and a fluid outlet 216 for discharging the biological fluid 204. In some cases, the biological fluid removal and filtering device 200 further includes a catheter 208 the biological fluid 204 within the patient. In some cases, the biological fluid removal and filtering device 200 is fluidically coupled to an in-dwelling catheter to remove the biological fluid 204.

In some cases, the fluid inlet 212 and the fluid outlet 216 include Luer-Lock connections so that the biological filtering device 210 can easily be included in existing kits for biological fluid 204 removal (e.g., thoracentesis kits). In some cases, the biological filtering device 210 includes a pressure relief valve that, when opened, can reverse flow of biological fluid 204 during a biological fluid removal procedure. In some cases, the pressure relief valve is fluidically attached upstream of the biological filtering device 210. In some cases, the pressure relief valve is fluidically attached downstream of the biological filtering device 210. In some cases, a filter (e.g., human cell filter 220 and/or bacteria cell filter 230) is attached to the pressure relief valve. In some cases, the biological fluid removal and filtering device 200 further includes a one-way valve preventing fluid from re-entering the patient. In some cases, the pressure relief valve is a one-way valve that prevents fluid from re-entering the patient.

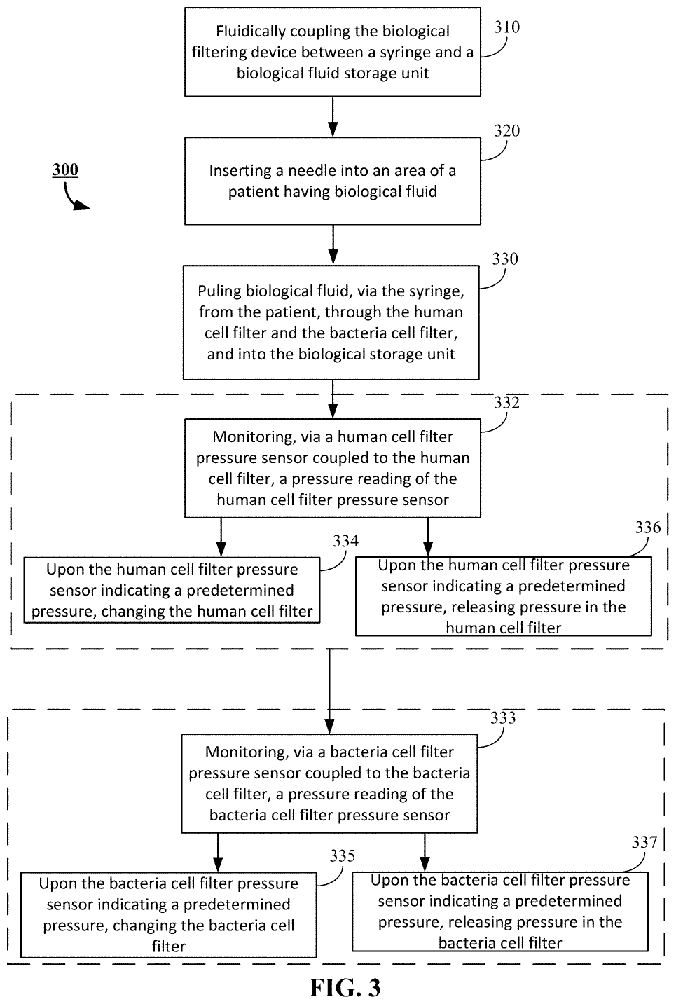

FIG. 3 illustrates a method of using a biological fluid removal and filtering device. Referring to FIGS. 2 and 3, a method 300 of using a biological filtering device 210 includes fluidically coupling (310) the biological filtering device 210 between a syringe 202 and a biological fluid storage unit 206, inserting (320) a catheter 208 into an area of a patient having biological fluid 204, and pulling (330) biological fluid, via the syringe 202, from the patient, through the human cell filter 220 and the bacteria cell filter 230, and into the biological storage unit 206. In this way, the desired human and bacteria cells are collected on the filter membranes of the corresponding human cell filter 220 and bacteria cell filter 230, which can subsequently be sent for microbiological analysis. In some cases, pulling (330) the biological fluid from the patient includes creating fluidic pressure, via the syringe, that pulls the biological fluid from the patient. In some cases, the biological fluid is pulled (330) from the patient by a gravitational force. In some cases, the biological fluid is pulled (330) from the patient by a pump.

In some cases, the method 300 further includes monitoring (332), via a human cell filter pressure sensor coupled to the human cell filter 220, a pressure reading of the human cell filter pressure sensor. In some cases, the method 300 further includes, upon the human cell filter pressure sensor indicating a predetermined pressure, changing (334) the human cell filter 220. For example, a user may remove the human cell filter gasket, uncouple the upstream end and the downstream of the human cell filter casing, and replace the human cell filter membrane with a new human cell filter membrane. In some cases, the method 300 further includes, upon the human cell filter pressure sensor indicating a predetermined pressure, releasing (336) pressure in the human cell filter 220 via a pressure relief valve. In some cases, releasing (336) pressure in the human cell filter includes reversing the flow of the biological fluid 204. In some cases, the predetermined pressure is a value that is equal to or exceeds a value that is known to correlate to a human cell filter membrane that is substantially full and/or clogged with biological material (e.g., human cells).

In some cases, the method 300 further includes monitoring (333), via a bacteria cell filter pressure sensor coupled to the bacteria cell filter 230, a pressure reading of the bacteria cell filter pressure sensor. In some cases, the method 300 further includes, upon the bacteria cell filter pressure sensor indicating a predetermined pressure, changing (335) the bacteria cell filter 230. For example, a user may remove the human cell filter gasket, uncouple the upstream end and the downstream of the bacteria cell filter casing, and replace the bacteria cell filter membrane with a new bacteria cell filter membrane. In some cases, the method 300 further includes, upon the bacteria cell filter pressure sensor indicating a predetermined pressure, releasing (337) pressure in the bacteria cell filter 230 via a pressure relief valve. In some cases, releasing (337) pressure in the human cell filter includes reversing the flow of the biological fluid 204. In some cases, the predetermined pressure is a value that is equal to or exceeds a value that is known to correlate to a bacteria cell filter membrane that is substantially full and/or clogged with biological material (e.g., bacteria cells). In some cases, the above described steps (e.g., 332, 333, 334, 335, 336, 337) may be carried out via the use of a single pressure sensor for the entire biological filtering device 210. In some cases, the above described steps may not be carried out with a pressure sensor at all; instead, trained users (e.g., technicians, nurses, physicians, and the like) may be able to “feel” when the pressure within the biological filtering device 210 has reached and/or exceeded a predetermined pressure (e.g., via resistance to the pressure created via the syringe 202).

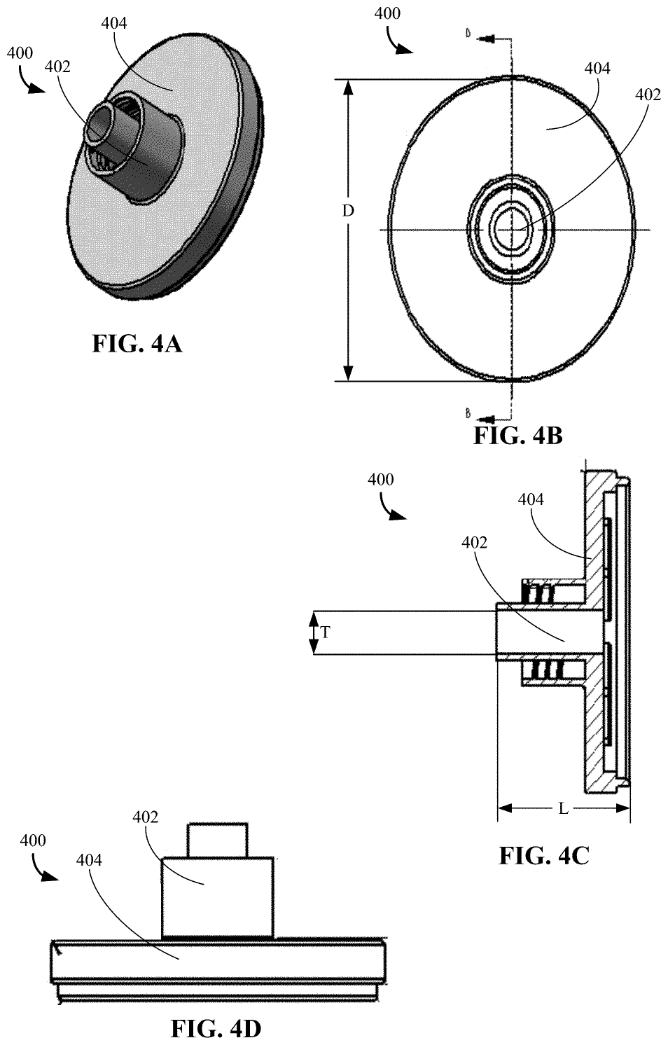

FIGS. 4A-4D illustrate an inlet side casing of a biological filtering device. Referring to FIGS. 4A-4D, an inlet side casing 400 of a biological filtering device includes a fluid inlet 402 and an upstream side 404 of a human cell filter casing. In some cases, the fluid inlet 402 includes a Luer-Lock connections (e.g., male or female). When the upstream side 404 of the human cell filter casing is combined with a downstream side of a human cell filter casing, a human cell filter gasket can provide a fluid-proof seal. In some cases, the fluid inlet 402 and the upstream side 404 of the human cell filter casing are separate pieces that are attached to one another; in other cases, the fluid inlet 402 and the upstream side 404 of the human cell filter casing are manufactured as a single piece. In some cases, the inlet side casing 400 includes a length (L) between 15 mm to 30 mm, a diameter (D) of 30 mm to 130 mm, and a wall thickness (T) of 6 mm.

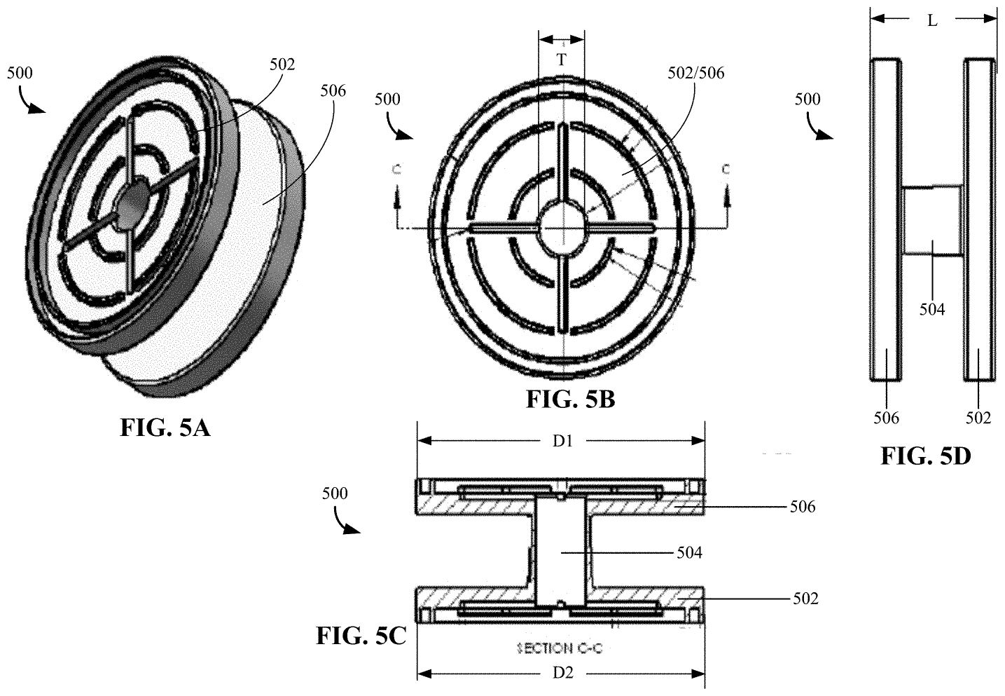

FIGS. 5A-5D illustrate a middle casing of a biological filtering device. Referring to FIGS. 5A-5D, a middle casing 500 of a biological filtering device includes a downstream side 502 of a human cell filter casing, a fluid connector 504 (e.g., for fluidically connecting a human cell filter and a bacteria cell filter) and an upstream side 506 of a bacteria cell filter casing. When the downstream side 502 of the human cell filter casing is combined with an upstream side (e.g., 404 of FIGS. 4A-4D) of a human cell filter casing, a human cell filter gasket can provide a fluid-proof seal. When the upstream side 506 of the bacteria cell filter casing is combined with the downstream side of the bacteria cell filter casing, a bacteria cell filter gasket can provide a fluid-proof seal. In some cases, the downstream side 502 of the human cell filter casing, the fluid connector 504, and the upstream side 506 of the bacteria cell filter casing are separate pieces that are attached to one another; in other cases, the downstream side 502 of the human cell filter casing, the fluid connector 504, and the upstream side 506 of the bacteria cell filter casing are manufactured as a single piece. This can make adding additional human cell filters and/or bacteria cell filters very simple. Indeed, a user merely needs to add additional middle casings between the fluid inlet and the fluid outlet (and each middle casings' corresponding filter membrane and gasket, with the pore size of each successive filter membrane getting smaller towards the fluid outlet). In some cases, the human cell filter casing and the bacteria cell filter casing may have different diameters (e.g., D1 and D2). In some cases, the middle casing 500 includes a length (L) between 30 mm to 60 mm, a diameter (D) of 30 mm to 130 mm, and a wall thickness (T) of 6 mm.

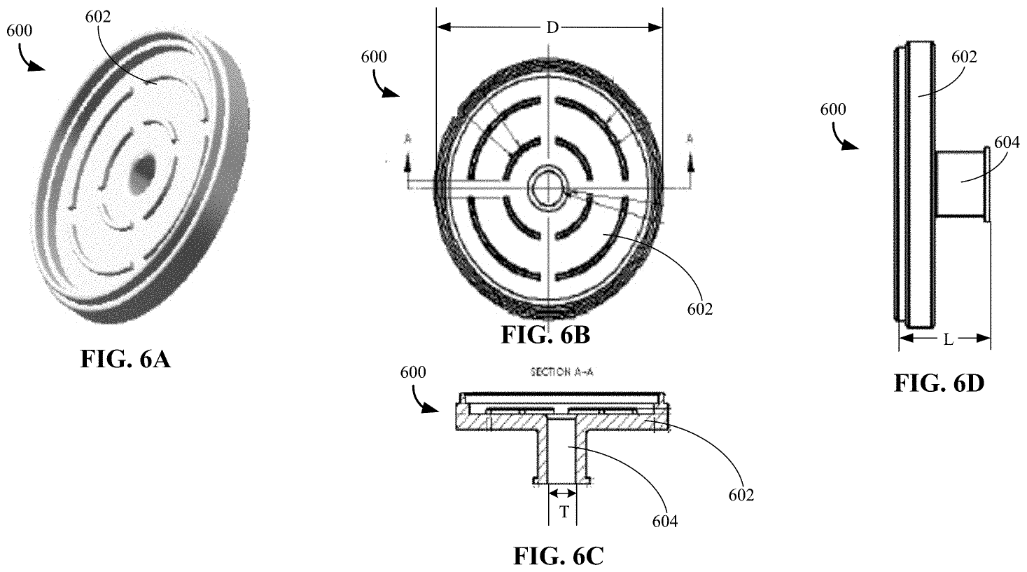

FIGS. 6A-6D illustrate an outlet side casing of a biological filtering device. Referring to FIGS. 6A-6D, an outlet side casing 600 of a biological filtering device includes a downstream side 602 of a bacteria cell filter casing and a fluid outlet 604. In some cases, the fluid outlet 604 includes a Luer-Lock connections (e.g., male or female). When the downstream side 602 of the bacteria cell filter casing is combined with an upstream side (e.g., 506 of FIGS. 5A-5D) of a bacteria cell filter casing, a bacteria cell filter gasket can provide a fluid-proof seal. In some cases, the fluid outlet 604 and the downstream side 602 of the bacteria cell filter casing are separate pieces that are attached to one another; in other cases, the fluid outlet 604 and the downstream side 602 of the bacteria cell filter casing are manufactured as a single piece. In some cases, the outlet side casing 600 includes a length (L) between 15 mm to 30 mm, a diameter (D1 and/or D2) of 30 mm to 130 mm, and a wall thickness (T) of 6 mm.

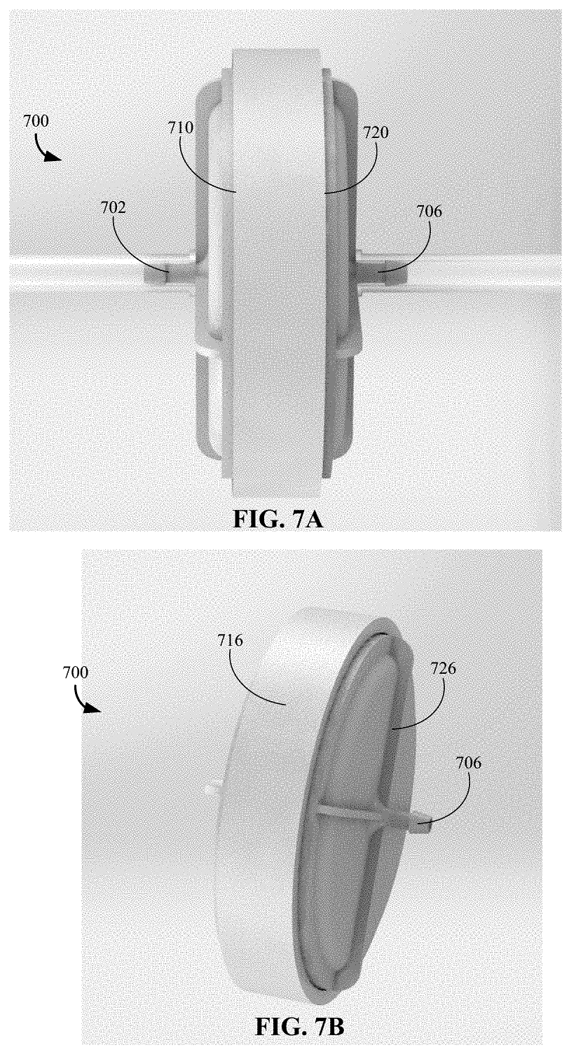

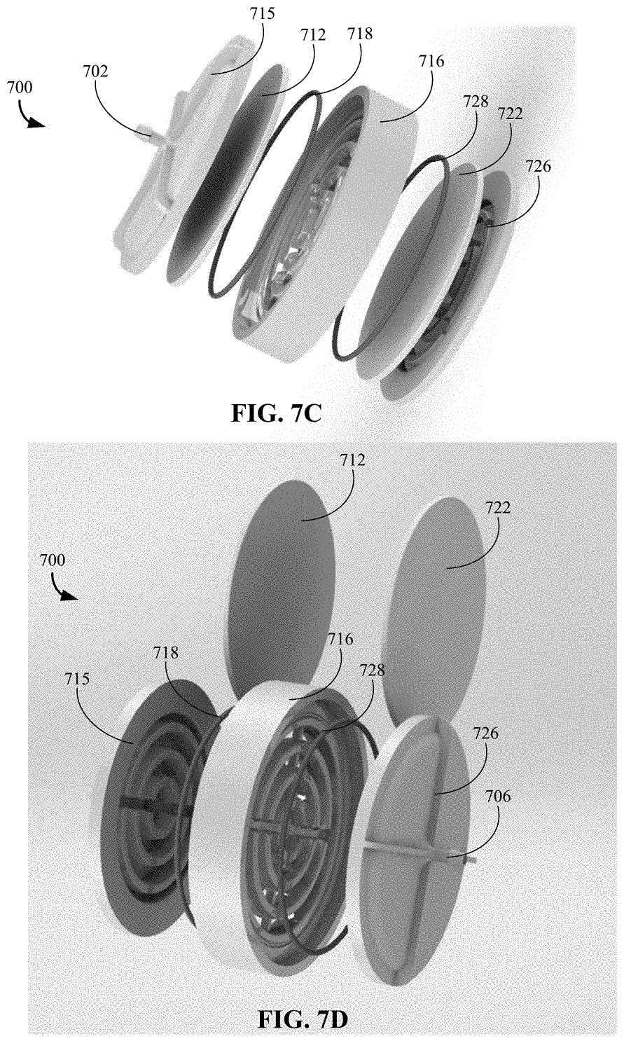

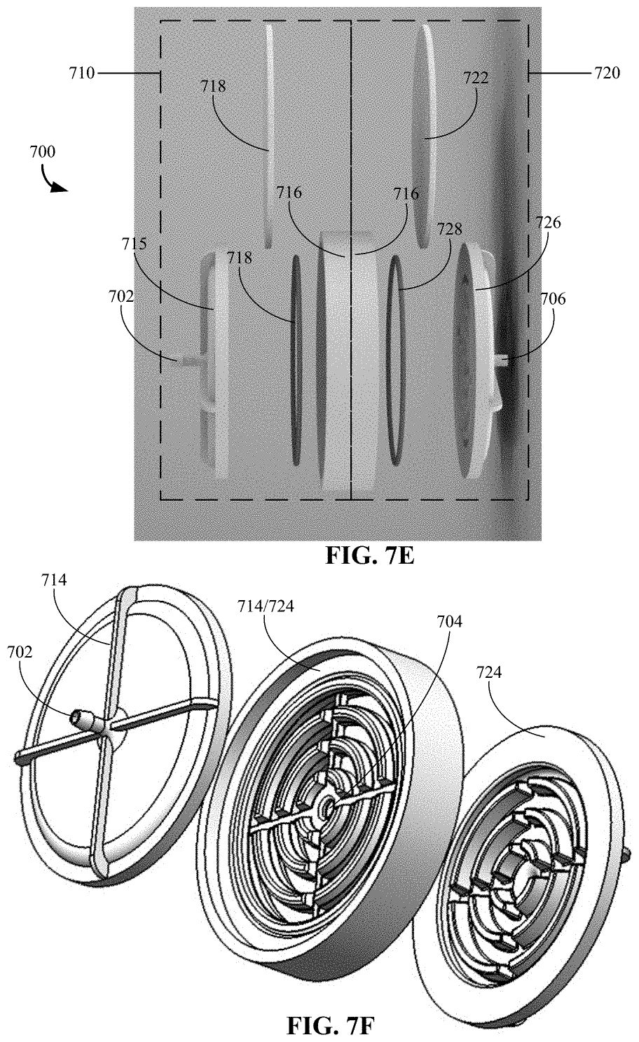

FIGS. 7A and 7B illustrate perspective views of a biological filtering device. FIGS. 7C, 7D, and 7E illustrate exploded views of a biological filtering device. FIG. 7F illustrates a perspective view of a casing of a biological filtering device. Referring to FIGS. 7A-7F, a biological filtering device 700 includes a fluid inlet 702 for receiving biological fluid, a human cell filter 710 coupled to the fluid inlet 702 and having a pore size to capture human cells, a bacteria cell filter 720 having a pore size to capture bacteria cells, a fluid connector 704 fluidically connecting the human cell filter 710 and the bacteria cell filter 720, and a fluid outlet 706 for discharging the biological fluid.

During use, the flow of biological fluid enters a biological filtering device 700 via a fluid inlet 702, flows through the biological filtering device 700, including the human cell filter 710 and the bacteria cell filter 720, and exits the biological filtering device 700 via a fluid outlet 706.

In some cases, as illustrated in FIGS. 7A-7F, the fluid connector 704 is physically (e.g., attached via structure and fluidically) coupled to the human cell filter 710 and the bacteria cell filter 720. In some cases, the biological filtering device 700 further includes a second human cell filter coupled to the fluid connector 704 between the human cell filter 710 and the bacteria cell filter 720. The pore size of the second human cell filter is smaller than the pore size of the human cell filter 710 (which is why the second human cell filter is positioned downstream of the human cell filter 710). In some cases, the biological filtering device 700 further includes a second bacteria cell filter coupled to the fluid connector 704 between the human cell filter 710 and the bacteria cell filter 720. The pore size of the second bacteria cell filter is larger than the pore size of the bacteria cell filter 720 (which is why the second bacteria cell filter is positioned upstream of the bacteria cell filter 720). In some cases, the biological filtering device 100 can include two or more of human cell filters and/or bacteria cell filters. Indeed, due to the structure of the casing for the filters, as many filters with as many different pore sizes as is desired can be added to the biological filtering device 700.

Referring specifically to FIGS. 7C-7F, in some cases, when assembled, both the human cell filter 710 and the bacteria cell filter 720 each include a filter membrane 712, 722, a casing 714, 724 formed around the filter membrane 712, 722, and a gasket 718, 728 fluidically sealing the casing 714, 724 around the filter membrane 712, 722. In other words, the gasket 718, 728 provides a fluid proof seal around its corresponding filter 710, 720. In some cases, a pressure relief valve is configured to reverse flow and/or release pressure of biological fluid during a biological fluid removal procedure, as explained in more detail above with respect to FIG. 2.

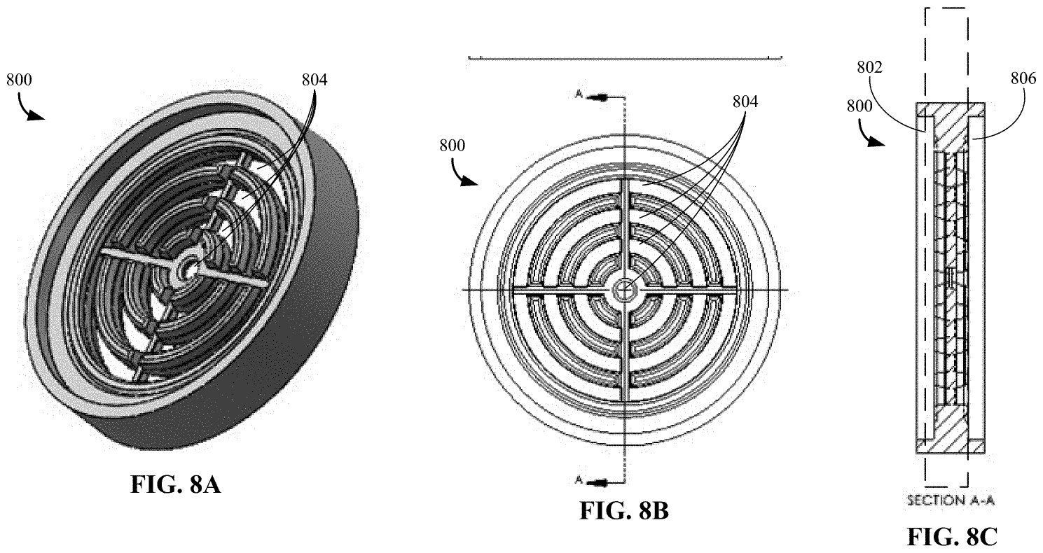

In some cases, the casing 714 of the human cell filter 710 includes an upstream end 715 that is attached to the fluid inlet 702 and a middle portion 716 that includes the fluid connector 704. In some cases, the casing 724 of the bacteria cell filter 720 includes the middle portion 716 that includes the fluid connector 704 and a downstream end 726 that is attached to the fluid outlet 706. In other words, the middle portion 716 acts as a downstream portion for the human cell filter 710 and an upstream portion for the bacteria cell filter 720. In this way, in order to add more human cell filters and/or more bacteria cell filters, the middle portion 716 (e.g., 800 as illustrated in FIGS. 8A-8C and that includes/acts as the fluid connector 704), a gasket 718 or 728, and an appropriately sized filter membrane is all that needs to be added between the fluid inlet 702 and the fluid outlet 706. This allows for great flexibility to have as many filters as is desired to filter cells from biological fluid.

In the case of multiple human cell filters and/or bacteria cell filters, each of the respective filter membranes may have a pore size falling within the above described ranges, with the larger pore size being upstream of the smaller pore size. Furthermore, the exact pore size of each filter membrane depends on the specific type of human and/or bacteria cells that are desired to be collected, based on the known sizes of those human and/or bacteria cells. It should be understood that the pore size of the human cell filter 710 and the bacteria cell filter 720 refers to the pore size of the filter membrane 712, 722 for that filter.

FIGS. 8A, 8B, and 8C illustrate a middle casing of a biological filtering device. Referring to FIGS. 8A-8C, a middle casing 800 of a biological filtering device includes a downstream side 802 of a human cell filter casing, a fluid connector 804 (e.g., for fluidically connecting a human cell filter and a bacteria cell filter) and an upstream side 806 of a bacteria cell filter casing. When the downstream side 802 of the human cell filter casing is combined with an upstream side (e.g., 715 of FIGS. 7A-7F) of a human cell filter casing, a human cell filter gasket can provide a fluid-proof seal. When the upstream side 806 of the bacteria cell filter casing is combined with the downstream side of the bacteria cell filter casing (e.g., 726 of FIGS. 7A-7F), a bacteria cell filter gasket can provide a fluid-proof seal.

As used herein, the term “subject” and “patient” are used interchangeably and refer to both human and nonhuman animals. The term “nonhuman animals” includes all vertebrates (e.g., mammals and nonmammals), such as nonhuman primates, sheep, dogs, cats, horses, cows, chickens, amphibians, reptiles, and the like.

As used herein, recitation of ranges are merely intended to serve as a shorthand method of referring individually to each separate value falling within the range, unless otherwise indicated, and each separate value is incorporated into the specification as if it were individually recited. For example, if a range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest and highest value enumerated are to be considered to be expressly stated in this disclosure.

Although the subject matter has been described in language specific to structural features and/or acts, it is to be understood that the subject matter defined in the appended claims is not necessarily limited to the specific features or acts described above. Rather, the specific features and acts described above are disclosed as examples of implementing the claims and other equivalent features and acts are intended to be within the scope of the claims.

What is claimed is:

1. A biological filtering device, comprising:
a fluid inlet for receiving a biological fluid;
a human cell filter coupled to the fluid inlet and having a pore size to capture human cells;
a bacteria cell filter having a pore size to capture bacteria cells;
a fluid connector fluidically connecting the human cell filter to the bacteria cell filter; and
a fluid outlet coupled to the bacteria cell filter for discharging the biological fluid.

2. The device of claim 1, wherein the pore size of the bacteria cell filter is equal to or greater than 0.1 microns and less than 0.51 microns.

3. The device of claim 1, wherein the pore size of the human cell filter is 10 equal to or greater than 2 microns and less than 15.1 microns.

4. The device of claim 1, wherein the human cell filter and the bacteria cell filter are hydrophilic.

5. The device of claim 1, wherein the fluid inlet and the fluid outlet include Luer-Lock connections.

6. The device of claim 1, wherein the fluid connector is coupled to the human cell filter and the bacteria cell filter.

7. The device of claim 1, further comprising:
a second human cell filter coupled to the fluid connector between the human cell filter and the bacteria cell filter and having a pore size to capture human cells, wherein the pore size of the second human cell filter is smaller than the pore size of the human cell filter.

8. The device of claim 1, further comprising:
a second bacteria cell filter coupled to the fluid connector between the human cell filter and the bacteria cell filter and having a pore size to capture bacteria cells, wherein the pore size of the second bacteria cell filter is larger than the pore size of the bacteria cell filter.

9. The device of claim 1, wherein the human cell filter and the bacteria cell filter each comprises:
a filter membrane;
a casing formed around the filter membrane; and
a gasket fluidically sealing the casing around the filter membrane.

10. The device of claim 1, further comprising a pressure release valve that is configured to reverse flow of biological fluid during a biological fluid removal procedure.

11. A biological fluid removal and filtering device, comprising:
a syringe for withdrawing biological fluid from a patient;
a biological filtering device fluidically coupled to the syringe, comprising:
a fluid inlet for receiving a biological fluid;
a human cell filter coupled to the fluid inlet and having a pore size to capture human cells;
a bacteria cell filter having a pore size to capture bacteria cells;
a fluid connector connecting the human cell filter to the bacteria cell filter; and
a fluid outlet coupled to the bacteria cell filter for discharging the biological fluid;
a biological fluid storage unit fluidically coupled to the biological filtering device.

12. The biological fluid removal and filtering device of claim 11, wherein during use, the syringe pulls biological fluid from a patient, through the human cell filter and the bacteria cell filter, and into the biological fluid storage unit.

13. The biological fluid removal and filtering device of claim 11, further comprising a catheter attached to the syringe, wherein the catheter is used to access biological fluid within a patient.

14. A method of using a biological filtering device, comprising:
fluidically coupling the biological filtering device between a syringe and a biological fluid storage unit, wherein the biological filtering device comprises:
a fluid inlet for receiving a biological fluid;
a human cell filter coupled to the fluid inlet and having a pore size to capture human cells;
a bacteria cell filter having a pore size to capture bacteria cells;
a fluid connector connecting the human cell filter to the bacteria cell filter; and
a fluid outlet coupled to the bacteria cell filter for discharging the biological fluid;
inserting a catheter into an area of a patient having biological fluid; and
pulling biological fluid, via the syringe, from the patient, through the human cell filter and the bacteria cell filter, and into the biological fluid storage unit.

15. The method of claim 14, wherein the biological filtering device further comprises a human cell filter pressure sensor coupled to the human cell filter, the method further comprising monitoring a pressure reading of the human cell filter pressure sensor.

16. The method of claim 15, further comprising, upon the human cell filter pressure sensor indicating a predetermined pressure, changing the human cell filter.

17. The method of claim 15, wherein the biological filtering device further comprises a pressure relief valve coupled to the human cell filter, the method further comprising, upon the human cell filter pressure sensor indicating a predetermined pressure, releasing pressure in the human cell filter via the pressure relief valve.

18. The method of claim 14, wherein the biological filtering device further comprises a bacteria cell filter pressure sensor coupled to the bacteria cell filter, the method further comprising monitoring a pressure reading of the bacteria cell filter pressure sensor.

19. The method of claim 18, further comprising, upon the bacteria cell filter pressure sensor indicating a predetermined pressure, changing the bacteria cell filter.

20. The method of claim 18, wherein the biological filtering device further comprises a pressure relief valve coupled to the bacteria cell filter, the method further comprising, upon the bacteria cell filter pressure sensor indicating a predetermined pressure, releasing pressure in the bacteria cell filter via the pressure relief valve.
